(19) United States
(12) Patent Application Publication
(10) Pub. No.: US 2012/0171121 A1
(43) Pub. Date: Jul. 5, 2012

(54) Rosette Nanotubes as Drug Delivery Agents

(75) Inventor: Thomas J. Webster, Barrington, RI (US)

(73) Assignee: Brown University, Providence, RI (US)

(21) Appl. No.: 13/342,479

(22) Filed: Jan. 3, 2012

Related U.S. Application Data
(60) Provisional application No. 61/429,660, filed on Jan. 4, 2011.

(51) Int. Cl. A61K 49/00; A61K 47/22; B82Y 5/00

(52) U.S. Cl. 424/9.1; 514/788; 977/788; 977/906

(57) ABSTRACT

The present invention is directed to delivery complexes of rosette nanotubes and one or more biologically active agents or diagnostic agents.

### RELATED APPLICATION DATA

This application claims priority to U.S. Provisional Patent Application No. 61/429,660, filed on Jan. 4, 2012 and is hereby incorporated herein by reference in its entirety for all purposes.

### STATEMENT OF GOVERNMENT INTERESTS

This invention was made with government support under National Institutes of Health grant number NIH R21AG027521. The Government has certain rights in the invention.

### FIELD

Embodiments of the present disclosure relate to the use of rosette nanotubes for the delivery of agents for therapeutic or diagnostic purposes. Embodiments of the present disclosure further relate to drug delivery complexes of rosette nanotubes and biologically active agents and compositions thereof and the use of such drug delivery complexes to deliver biologically active agents to individuals for therapeutic purposes. Embodiments of the present disclosure still further relate to diagnostic delivery complexes of rosette nanotubes and diagnostic agents and compositions thereof and the use of such diagnostic delivery complexes to deliver diagnostic agents to individuals for diagnostic purposes.

### BACKGROUND

There has been increasing interest in the use of injectable, biocompatible, biodegradable polymers and nanoparticles for anticancer drug delivery applications. Methods based on external stimuli (i.e., ultraviolet light for crosslinking) to create amphiphilic polymers or lipid drug mixtures to deliver water-insoluble anticancer drugs seek to improve drug solubility in water, increase drug bioavailability, and decrease drug toxicity. In addition, molecules with self-assembly properties have been studied for such applications. However, complex, chemical treatments used to create some of these drug carriers can result in a reduction of drug efficacy. Also, many simpler mixtures provide a fast, uneven release of hydrophobic drugs under physiological conditions, thus limiting prolonged cell-drug interactions.

There is a need in the art for complexes that can deliver biologically active agents and diagnostic agents to an individual for therapeutic and diagnostic purposes.

It is therefore an object of the present invention to create complexes of agents, such as biologically active agents and/or diagnostic agents, and rosette nanotubes that can be administered to individuals for therapeutic and/or diagnostic purposes. Such complexes are delivery vehicles for the biologically active agents and/or diagnostic agents. It is an additional object of the present invention to provide methods of treating individuals by administering the complexes of the present disclosure. These and other objects, features, and advantages of the invention or certain embodiments of the invention will be apparent to those skilled in the art from the following disclosure and description of exemplary embodiments.

### SUMMARY

Embodiments of the present disclosure are directed to complexes of a self-assembled rosette nanotube and one or more or a plurality of agents. Such agents include biologically active agents and/or diagnostic agents. The complexes are administered to an individual where the biologically active agent and/or diagnostic agent is delivered to a site within the individual, including into the cell of an individual, and is made available for therapeutic or diagnostic purposes. According to one aspect, the agent dissociates from the rosette nanotube to treat an individual or to provide a diagnostic capability. According to an additional aspect, the agent remains attached to, bound to, or complexed with or combined with the rosette nanotube.

According to one aspect, a delivery complex is produced by combining modules of a self-assembled rosette nanotube and one or more agents, such as therapeutic or diagnostic agents, in media where the modules self-assemble into a rosette nanotube which incorporates the one or more agents to form a complex of a rosette nanotube and the one or more agents. According to an additional aspect, a delivery complex is produced by combining a self-assembled rosette nanotube and one or more agents, such as therapeutic or diagnostic agents, in media whereupon the one or more agents are incorporated into the rosette nanotube to form a complex of a rosette nanotube and one or more agents. The delivery complex may then be administered to an individual for therapeutic or diagnostic purposes.

The modules may be any of those known to persons of ordinary skill in the art such as ĜC motifs, unmodified or modified to include moieties or sidechains, which self-assemble into helical rosette nanotubes. According to one embodiment, modules are placed into an aqueous medium where they self assemble into a substructure such as a ring structure, such as a rosette, and the ring structures then self-assemble by stacking one on top of another to form a tubular structure, commonly referred to as a nanotube. Such modules, substructures and nanometer scale molecular structures and their self-assembly is described in U.S. Pat. No. 6,696,565, Fenniri et al, J. Am. Chem. Soc. 2001, 123, 3854-3855, Moralez et al., J. Am. Chem. Soc., 2005, 127, 8307-8309, Fine et al., International Journal of Nanomedicine 2009:4 91-97; and Zhang et al., Biomaterials 2009; 30(7): 1309-1320 each of which are hereby incorporated by reference in their entireties for all purposes.

Rosette nanotubes of the present disclosure are very stable in water and lack virus-related safety concerns and toxicity at amounts of about 1 μg/ml. See Int. J. Nanomedicine, 2008, 3(3):373-383; Small. 2008, 4(6):817-823; and Am. J. Physiol Lung Cell Mol. Physiol. 2005, November; 289(5):L698-708 each of which are hereby incorporated by reference in their entireties.

According to one aspect of the present disclosure, methods are provided where the self-assembly of modules incorporates the agent into or otherwise complexes the agent with, the self-assembled rosette nanotube. According to another aspect, fully assembled rosette nanotubes can be incubated with one or more or a plurality of agents and the one or more or plurality of agents can complex with or otherwise bind to, the fully assembled rosette nanotube to form a composite. According to one further aspect, the one or more or plurality of agents are joined to or bound to the self-assembled rosette nanotube through steric, ionic, van der Waals, dispersion or other noncovalent interactions to form a rosette nanotube and agent complex useful as a therapeutic or diagnostic delivery agent and in some cases in the preparation of a pharmaceutical agent to be administered to an individual. According to an additional further aspect, the one or more agents are covalently attached by methods known to those of skill in the art to the rosette nanotube to form a rosette nanotube and agent complex useful as a therapeutic or diagnostic delivery agent and in some cases in the preparation of a pharmaceutical agent to be administered to an individual.

According to certain aspects, rosette nanotubes are functionalized with one or more biologically active agents to form a complex, for example tamoxifen or dexamethasone bound to the rosette nanotube. The complex is administered to an individual for therapeutic purposes. In this aspect, the rosette nanotube is a delivery vehicle or carrier for the biologically active agent.

According to certain aspects, rosette nanotubes are functionalized with one or more diagnostic agents to form a complex, for example radiolabeled agents bound to the rosette nanotube. The complex is administered to an individual for diagnostic purposes. In this aspect, the rosette nanotube is a delivery vehicle or carrier for the diagnostic agent.

Embodiments of the present invention are still further directed to compositions including rosette nanotube/agent complexes used as a vehicle for the delivery of the agent to or into a particular cell. According to certain embodiments, the rosette nanotube and agent complexes are mixed with a pharmaceutically acceptable excipient or delivery vehicle and then delivered to the desired location within an individual. In addition, delivery vehicle kits are provided that include the rosette nanotubes of the present invention for complexing with one or more desired agents using the methods described herein pursuant to instructions and optional reagents provided in the kit to form a delivery reagent for a biologically active agent or diagnostic agent.

### BRIEF DESCRIPTION OF THE DRAWINGS

FIG. 1 shows the structure of an exemplary module used to form a rosette nanotube. Shown in schematic form is a rosette nanotube and also shown is an image of rosette nanotubes of the present disclosure.

FIG. 2 UV-Vis spectra recorded in methanol of A) TAM (5 μg/mL), B) K1 (left) and TBL (right) (25 μg/mL), C) TAM-K1 (left) and TAM-TBL (right) (25 μg/mL).

FIG. 3 UV-Vis spectra recorded in water of A) TAM-K 1 (left) and TAM-TBL (right), and B) K1 (left) and TBL (right). All samples were at a concentration of 25 μg/mL.

FIG. 4 Tapping mode AFM height images of A) K1, B) TAM-K1, C) TBL, and D) TAM-TBL.

FIG. 5 Quantitative height measurements by atomic force microscopy. Data are mean±standard error of mean (more than 30 random points were chosen from atomic force microscopy scans). *P<0.05 compared with K1 only; #P<0.05 compared with TBL only.

FIG. 6 Tapping mode AFM height profiles of (A) RNTs and (B) RNT-encapsulated DEX. Average heights measured for the RNTs and the RNT-DEX complex were 2.91±0.95 nm and 7.04±0.33 nm, respectively.

FIG. 7 Comparison of UV-vis absorbance curves in methanol (Left) and water (Right). (A) 0.005 mg/mL DEX, (B) 0.025 mg/mL DEX-RNTs and (C) 0.025 mg/mL RNTs. (NOTE: There is no UV absorbance curve of DEX only in water, because DEX precipitates in water.)

FIG. 8 DEX release curve up to 9 days. Data are mean±SEM (n=12). *p<0.05 compared to DEX concentrations at day 1 to 5. **p<0.05 compared to DEX adsorbed on glass slides at respective days. ***p<0.05 compared to DEX adsorbed on glass slides and DEX adsorbed on RNT coated glass slides.

FIG. 9 Osteoblast density cultured with released DEX. Data are mean±SEM (n=9). **p<0.05 compared to negative controls (no additives). *p<0.05 compared to both controls and DEX released from glass slides at day 5. #p<0.05 compared to day 1 and 3 results on respective samples.

### DETAILED DESCRIPTION OF CERTAIN EXEMPLARY EMBODIMENTS

The aspects, advantages and other features of the disclosure will become apparent in view of the following detailed description, which discloses various non-limiting embodiments of the disclosure. In describing embodiments of the present disclosure, specific terminology is employed for the sake of clarity. However, the disclosure is not intended to be limited to the specific terminology so selected. It is to be understood that each specific element includes all technical equivalents that operate in a similar manner to accomplish a similar purpose. Additionally, all of the citations herein are incorporated by reference in their entirety.

Embodiments of the present disclosure involve administering biologically active agents or diagnostic agents to an individual using a complex of the agent and rosette nanotubes for therapeutic or diagnostic purposes. In particular, disclosed herein are novel transport complexes, comprising an unexpectedly advantageous transport vehicle. The rosette nanotube is a carrier that is formed from self-assembled modules as described below and those modules recognized in the art.

Modules according to the present disclosure include compounds of Formula I below:

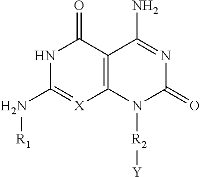

wherein X is CH or nitrogen; n is an integer of, 1, 2, 3, or 4; R₂ is hydrogen or a linker group for example (CH₂), or other linker groups described herein; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such as alkyl, straight or branched chain, saturated or unsaturated; and salts thereof. Preferably R₁ is C₁ to C₁₀ alkyl, C₁ to C₅ alkyl, C₁ to C₃ alkyl, or methyl. Compounds within the scope of the invention include those where the Y group can be connected to the linker group either by the amino group or the carboxyl group of the amino acid or polypeptide. An exemplary linker group is shown in the formula below.

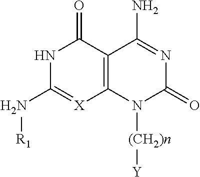

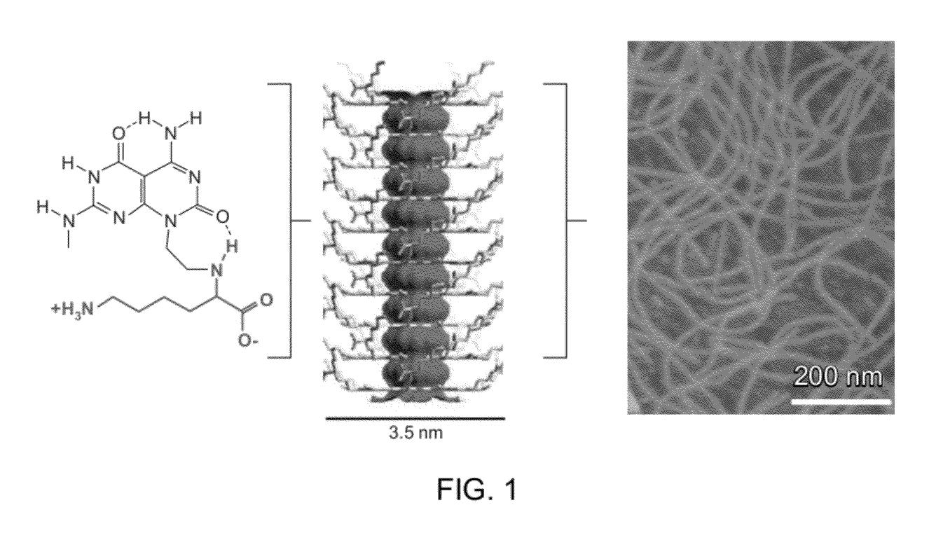

An exemplary module within the scope of formula I is shown in FIG. 1 along with a schematic representation of a nanotube and an image of nanotubes formed from the exemplary module.

Alternative linker groups R₂ can join the Y group to the carbon of the (CH₂)ₙ group or the N atom either by the amino group or the carboxyl group of the amino acid or polypeptide.

Alternative Linker moieties within the scope of the present disclosure include NH₃⁺ and the following

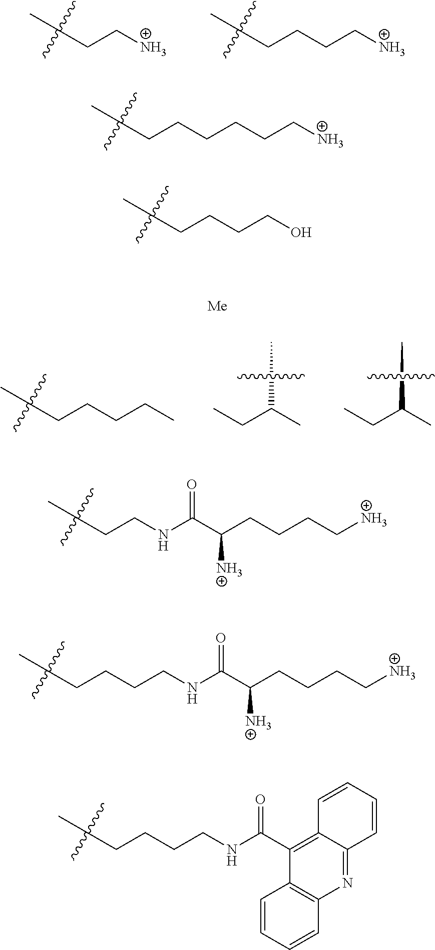

Compounds of Formula I can be prepared by the methods described in U.S. Pat. No. 6,696,565 hereby incorporated by reference herein in its entirety alone or combined with methods known to those of skill in the art.

Modules according to the present disclosure also include compounds of Formula II below:

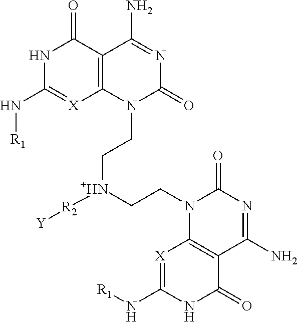

wherein X is CH or nitrogen; R₂ is hydrogen or a linker group for example (CH₂)ₙ where n is an integer of, 1, 2, 3, or 4 or (CH₂)₃CO other linker groups described herein; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such alkyl, straight or branched chain, saturated or unsaturated; and salts thereof. Preferably R₁ is C₁ to C₁₀ alkyl, C₁ to C₅ alkyl, C₁ to C₃ alkyl, or methyl. An exemplary linker group is shown in the formula below.

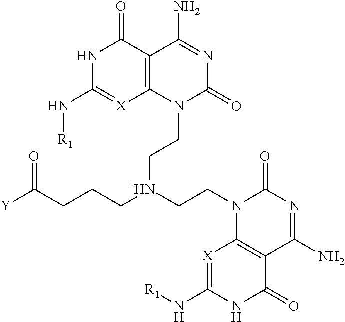

Compounds within the scope of the present disclosure include those where the Y group can be connected to the linker group either by the amino group or the carboxyl group of the amino acid or polypeptide. Alternative linker groups R₂ connecting the NH⁺ group and the Y group include

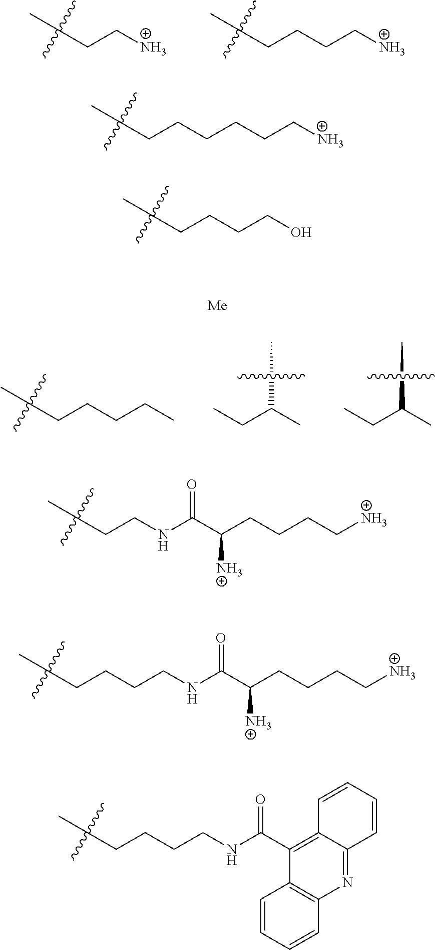

According to certain aspects of the present disclosure, the structure of Formula II is referred to as a twin base with a linker (TBL) or twin base linkers insofar as two similar double ring structures are present as shown in Formula II and are linked to an amino acid or polypeptide. However, it is to be understood that the two double ring structures need not be identical insofar as they may have different X and R₁ groups.

The term “amino acid” is inclusive of the 20 common amino acids, as well as “nonstandard amino acids,” for example, D-amino acids and chemically (or biologically) produced derivatives of “common” amino acids, including for example, β-amino acids. Accordingly, amino acids according to the present disclosure include the commonly known amino acids such as glycine (Gly, G), alanine (Ala, A), valine (Val, V), leucine (Leu, L), isoleucine (Ile, I), proline (Pro, P), hydroxyproline, phenylalanine (Phe, F), tyrosine (Tyr, Y), tryptophan (Trp, W) cysteine (Cys, C), methionine (Met, M) serine (Ser, S), o-phosphoserine, threonine (Thr, T), lysine (Lys, K), arginine (Arg, R), histidine (His, H), aspartate (Asp, D), glutamate (Glu, E), γ-carboxyglutamate, asparagine (Asn, N), glutamine (Gln, Q) and the like. Amino acids also include stereoisomers thereof and compounds structurally similar to the amino acids or modifications or derivatives thereof. Exemplary amino acids within the scope of the present disclosure include lysine, arginine, serine, glycine, aspartate and the like.

The term “peptide” is inclusive of both straight and branched amino acid chains, as well as cyclic amino acid chains, which comprise at least 2 amino acid residues. The terms “peptide” and “polypeptide” are used interchangeably herein. Accordingly, polypeptides according to the present disclosure include two or more amino acids covalently linked together. According to one aspect, the two or more amino acids are covalently linked together at least in part by one or more peptide bonds.

According to aspects of the present disclosure, modules (compounds) according to Formula I and Formula II self-assemble into substructures also called supermacrocycles which themselves will self-assemble into nanometer scale architectures or structures such as discrete nanotubular assemblies in water or aqueous solutions. Supermacrocycles are defined herein as being a number of organic molecules covalently or noncovalently bound together so as to form a ring structure. For example, compounds of Formula I will self-assemble into a 6-mer ring structure, sometimes referred to as a rosette. The process of forming nanotubes with the modules of the present disclosure is hierarchical. In particular, the modules of the present invention first self-assemble into supermacrocycles, and then the supermacrocycles self-assembly into nanotubes. Such self-assembly is described in U.S. Pat. No. 6,696,565. For the compounds of Formula II referred to as twin base linkers, the compounds will also assemble into a 6-mer ring structure. However, a single supermacrocycle formed will include two base layers owing to the presence of the two bases in each of the compound of Formula II.

According to preferred aspects of the present disclosure, the compounds of Formula I and Formula II include low molecular weight synthetic DNA base analogues referred to by the nomenclature ĈG. See Fenniri et al, J. Am. Chem. Soc. 2001, 123, 3854-3855. The ĈG moiety, referred to as a single ĈG motif, possess the Watson-Crick donor-donor-acceptor of guanine and the acceptor-acceptor-donor of cytosine and undergoes a self-assembly process, fueled by an array of hydrogen bonds, to produce a six-membered supermacrocycle or rosette. Stacking of these rosettes produced a nanotube of very high aspect ratio. Compounds within the scope of the present invention include a twin ĜC motif denoted as (ĈG)₂. Like the single ĈG motif, the twin ĈG motif (ĈG)₂ also possesses the Watson-Crick donor-donor-acceptor of guanine and the acceptor-acceptor-donor of cytosine and undergoes a self assembly process, fueled by an array of hydrogen bonds, to produce a six-membered supermacrocycle or ring structure (rosette) of twin configuration. Stacking of these twin rosettes produces a nanotube of very high aspect ratio and higher stability.

It should be understood that the above described Formula I and Formula II demonstrate that electrostatic, stacking and hydrophobic interactions can be effectively orchestrated by hydrogen bonds to direct the hierarchical assembly and organization of helical nanotubular architectures in an aqueous milieu. Helical nanotubular architectures within the scope of the present invention include those formed entirely from compounds of Formula I. Helical nanotubular architectures within the scope of the present invention include those formed entirely from compounds of Formula II. Further, helical nanotubular architectures within the scope of the present invention include those formed from one or more of the compounds of Formula I and one or more of the compounds of Formula II. For example, a supermacrocycle ring substructure having particular amino acid or polypeptide side chains formed from the compounds of Formula I can be stacked with a supermacrocycle ring substructure having particular amino acid or polypeptide side chains formed from compounds of Formula II. The rosette substructures formed from the compounds of Formula I and Formula II can be stacked in any desired sequence to form nanotubular structures of the present invention. Utilizing this aspect of the present invention, a wide variety of structurally different modules (i.e. molecules) can be synthesized and self-assembled into supermacrocycles and then nanotubular structures according to methods of the present invention.

According to certain aspects of the present disclosure, nanotubes range in lengths between about 1 nm and about 999 microns, about 1 nm to about 500 nm, about 10 nm to about 300 nm, or about 20 nm to about 100 nm. The nanotubes range in diameters between about 1 angstrom and about 100 nm, about 1 nm to about 30 nm, or from about 3 nm to about 15 nm. The openings or inner diameters through the nanotubes range in diameters between about 1 angstrom and about 100 nm, about 1 nm to about 30 nm, or from about 3 nm to about 15 nm. According to certain embodiments, the opening or inner diameter through the nanotube has a diameter of about 1 nm. According to certain embodiments, the nanotubes formed from the twin base linkers of formula II have a different opening or inner diameter compared to nanotubes formed from the compounds of formula I. This aspect allows for the incorporation into the nanotube of different sizes of agents, such as biologically active or diagnostic agents.

According to certain preferred aspects of the present invention, a nanotube is prepared from single base ring structures and twin base ring structures in any desired order. The nanotube can have one or more single base ring structures and one or more twin base ring structures. Likewise, a nanotube within the scope of the present invention can include a plurality of single base ring structures formed from compounds of Formula I and a plurality of twin base ring structures formed from compounds of Formula II stacked together, i.e. one next to the other via hydrogen bonding, to form the nanotube.

As may be used herein, the terms “drug,” biologically active agent,” and “therapeutic agent” are used interchangeably and are intended to include, but are not limited to, those compounds recognized by persons of skill in the art as being biologically active agents, or drugs or therapeutic agents and include any synthetic or natural element or compound which when introduced into the body causes a desired biological response, such as altering body function. Non-limiting examples of drugs or biologically active agents or therapeutic agents include chemotherapeutic agents, antiproliferative agents, cytotoxic agents and immunosuppressive agents and include molecules such as taxol, toxorubicin, daunorubicin, vinca-alcaoide, actinomycin, and tamoxifen. Further nonlimiting examples of drugs or biologically active agents or therapeutic agents include peptides (such as RGD, KRSR, YIGSR, IKVAV and the like), aromatic bioactive molecules such as tamoxifen, dexamethasone, vitamin K and the like, antibiotics such as penicillin, streptomycin, gentamycin and the like, and proteins such as bone morphogenetic proteins, matrillins and the like. Drugs or biologically active agents or therapeutic agents may be hydrophobic or hydrophilic. According to one aspect, the rosette nanotubes include hydrophobic moieties within the core portion of the structure where hydrophobic drugs, biologically active agents or therapeutic agents may be located in the composite. According to another aspect, the rosette nanotubes of the present disclosure may have hydrophilic outer surfaces to facilitate administration of the complexes in physiological environments.

Examples of anti-cancer drugs within the scope of the present disclosure that can be complexed with rosette nanotubes include bortezomib ([(1R)-3-methyl-1-[[(2S)-1-oxo-3-phenyl-2-[(pyrazinylcarbonyl)amino]propyl]amino]butyl]boronic acid; MG-341; VELCADE®), MG-132 (N-[(phenylmethoxy)carbonyl]-L-leucyl-N-[(1S)-1-formyl-3-methylbutyl]-leucinamide); pyrimidine analogs (e.g., 5-fluorouracil, floxuridine, capecitabine, gemcitabine and cytarabine); purine analogs; folate antagonists and related inhibitors (e.g., mercaptopurine, thioguanine, pentostatin and 2-chlorodeoxyadenosine [cladribine]); folic acid analogs (e.g., methotrexate); antimitotic agents, including vinca alkaloids (e.g., vinblastine, vincristine, and vinorelbine) and alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); microtubule disruptors (e.g., paclitaxel, docetaxel, vincristin, vinblastin, nocodazole, epothilones and navelbine, and teniposide); actinomycin, amsacrine, anthracyclines, bleomycin, busulfan, camptothecin, carboplatin, chlorambucil, cisplatin, cyclophosphamide, Cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP 16); dactinomycin (actinomycin D), daunorubicin, doxorubicin (adriamycin), idarubicin, anthracyclines, mitoxantrone, bleomycins, plicamycin (mithramycin) and mitomycin; L-asparaginase; antiplatelet agents; platinum coordination complexes (e.g., cisplatin, carboplatin), procarbazine, hydroxyurea, mitotane, aminoglutethimide; hormones and hormone analogs (e.g., estrogen, tamoxifen, goserelin, bicalutamide, nilutamide); aromatase inhibitors (e.g., letrozole, anastrozole); anticoagulants (e.g., heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (e.g., breveldin); immunosuppressives (e.g., cyclosporine, tacrolimus (FK-506), sirolimus (rapamycin), azathioprine, mycophenolate mofetil); anti-angiogenic compounds (e.g., TNP-470, genistein) and growth factor inhibitors (e.g., vascular endothelial growth factor (VEGF) inhibitors, fibroblast growth factor (FGF) inhibitors, epidermal growth factor (EGF) inhibitors); angiotensin receptor blockers; nitric oxide donors; anti-sense oligonucleotides; antibodies (e.g., trastuzumab (HERCEPTIN®), AVASTIN®, ERBITUX®); cell cycle inhibitors and differentiation inducers (e.g., tretinoin); mTOR (mammalian target of rapamycin) inhibitors (e.g., everolimus, sirolimus); topoisomerase inhibitors (e.g., doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan); corticosteroids (e.g., cortisone, dexamethasone, hydrocortisone, methylpednisolone, prednisone, and prenisolone); growth factor signal transduction kinase inhibitors; mitochondrial dysfunction inducers; and caspase activators and the like.

Examples of anti-cancer drugs within the scope of the present disclosure that can be complexed with rosette nanotubes include alemtuzumab; aminoglutethimide; amsacrine; anastrozole; asparaginase; bevacizumab; bicalutamide; bleomycin; bortezomib; buserelin; busulfan; campothecin; capecitabine; carboplatin; carmustine; CeaVac; cetuximab; chlorambucil; cisplatin; cladribine; clodronate; colchicine; cyclophosphamide; cyproterone; cytarabine; dacarbazine; daclizumab; dactinomycin; daunorubicin; dienestrol; diethylstilbestrol; docetaxel; doxorubicin; edrecolomab; epirubicin; epratuzumab; erlotinib; estradiol; estramustine; etoposide; exemestane; filgrastim; fludarabine; fludrocortisone; fluorouracil; fluoxymesterone; flutamide; gemcitabine; gemtuzumab; genistein; goserelin; huJ591; hydroxyurea; ibritumomab; idarubicin; ifosfamide; IGN-101; imatinib; interferon; irinotecan; ironotecan; letrozole; leucovorin; leuprolide; levamisole; lintuzumab; lomustine; MDX-210; mechlorethamine; medroxyprogesterone; megestrol; melphalan; mercaptopurine; mesna; methotrexate; mitomycin; mitotane; mitoxantrone; mitumomab; nilutamide; nocodazole; octreotide; oxaliplatin; paclitaxel; pamidronate; pentostatin; pertuzumab; plicamycin; porfimer; procarbazine; raltitrexed; rituximab; streptozocin; sunitinib; suramin; tamoxifen; temozolomide; teniposide; testosterone; thalidomide; thioguanine; thiotepa; titanocene dichloride; topotecan; tositumomab; trastuzumab; tretinoin; vatalanib; vinblastine; vincristine; vindesine; and vinorelbine and the like.

Examples of anticonvulsants within the scope of the present disclosure that can be complexed with rosette nanotubes include barbiturates (e.g., mephobarbital and sodium pentobarbital); benzodiazepines, such as alprazolam (XANAX®), lorazepam, clonazepam, clorazepate dipotassium, and diazepam (VALIUM®); GABA analogs, such as tiagabine, gabapentin (an α2δ antagonist, NEURONTIN®), and β-hydroxypropionic acid; hydantoins, such as 5,5-diphenyl-2,4-imidazolidinedione (phenyloin, DILANTIN®) and fosphenyloin sodium; phenyltriazines, such as lamotrigine; succinimides, such as methsuximide and ethosuximide; 5H-dibenzazepine-5-carboxamide (carbamazepine); oxcarbazepine; divalproex sodium; felbamate, levetiracetam, primidone; zonisamide; topiramate; and sodium valproate.

Examples of NMDA receptor antagonists within the scope of the present disclosure that can be complexed with rosette nanotubes include LY 274614 (decahydro-6-(phosphonomethyl)-3-isoquinolinecarboxylic acid), LY 235959 [(3S,4aR,6S,8aR)-decahydro-6-(phosphonomethyl)-3-isoquinolinecarboxylic acid], LY 233053 ((2R,4S)-rel-4-(1H-tetrazol-5-yl-methyl)-2-piperidine carboxylic acid), NPC 12626 (α-amino-2-(2-phosphonoethyl)-cyclohexanepropanoic acid), reduced and oxidized glutathione, carbamathione, AP-5 (5-phosphono-norvaline), CPP (4-(3-phosphonopropyl)-2-piperazine-carboxylic acid), CGS-19755 (seifotel, cis-4(phono-methyl)-2-piperidine-carboxylic acid), CGP-37849 ((3E)-2-amino-4-methyl-5-phosphono-3-pentenoic acid), CGP 39551 ((3E)-2-amino-4-methyl-5-phosphono-3-pentenoic acid, 1-ethyl ester), SDZ 220-581 [(αS)-α-amino-2′-chloro-5-(phosphonomethyl)-[1,1′-biphenyl]-3-propanoic acid], and S-nitrosoglutathione. amantadine, aptiganel (CERESTAT®, CNS 1102), caroverine, dextrorphan, dextromethorphan, fullerenes, ibogaine, ketamine, lidocaine, memantine, dizocilpine (MK-801), neramexane (MRZ 2/579, 1,3,3,5,5-pentamethyl-cyclohexanamine), NPS 1506 (delucemine, 3-fluoro-γ-(3-fluorophenyl)-N-methyl-benzenepropanamine hydrochloride), phencyclidine, tiletamine and remacemide. acamprosate, arcaine, conantokin-G, eliprodil (SL 82-0715), haloperidol, ifenprodil, traxoprodil (CP-101,606), and Ro 25-6981 [(±)-(R,S)-α-(4-hydroxyphenyl)-β-methyl-4-(phenylmethyl)-1-piperidine propanol]; aminocyclopropanecarboxylic acid (ACPC), 7-chlorokynurenic acid, D-cycloserine, gavestinel (GV-150526), GV-196771A (4,6-dichloro-3-[(E)-(2-oxo-1-phenyl-3-pyrrolidinylidene)methyl]-1′-1-indole-2-carboxylic acid monosodium salt), licostinel (ACEA 1021), MRZ-2/576 (8-chloro-2,3-dihydropyridazino[4,5-b]quinoline-1,4-dione 5-oxide 2-hydroxy-N,N,N-trimethyl-ethanam in ium salt), L-701,324 (7-chloro-4-hydroxy-3-(3-phenoxyphenyl)-2(1H)-quinolinone), HA-966 (3-amino-1-hydroxy-2-pyrrolidinone), and ZD-9379 (7-chloro-4-hydroxy-2-(4-methoxy-2-methylphenyl)-1,2,5,10-tetra-hydropyridanizo[4,5-b]quinoline-1,10-dione, sodium salt); oxidized and reduced glutathione, S-nitrosoglutathione, sodium nitroprusside, ebselen, and disulfuram, DETC-MeSO, carbamathione; CNQX (1,2,3,4-tetrahydro-7-nitro-2,3-dioxo-6-quinoxalinecarbonitrile) and DNQX (1,4-dihydro-6,7-dinitro-2,3-quinoxalinedione) and the like.

Examples of subtype-specific NMDA receptor antagonists within the scope of the present disclosure that can be complexed with rosette nanotubes include arcaine, argiotoxin636, Co 101244 (PD 174494, Ro 63-1908, 1-[2-(4-hydroxyphenoxy)ethyl]-4-[(4-methylphenyl)methyl-4-piperidinol], despiramine, dextromethorphan, dextrorphan, eliprodil, haloperidol, ifenprodil, memantine, philanthotoxin343, Ro-25-6981 ([(±)-(R*, S*)-α-(4-hydroxyphenyl)-β-methyl-4-(phenylmethyl)-1-piperidine propanol]), traxoprodil (CP-101,606), Ro 04-5595 (1-[2-(4-chlorophenyl)ethyl]-1,2,3,4-tetrahydro-6-methoxy-2-methyl-7-isoquinolinol), CPP [4-(3-phosphonopropyl)-2-piperazinecarboxylic acid], conantokin G, spermine, spermidine, NVP-AAM077 [[[[(1S)-1-(4-bromophenyl)ethyl]amino](1,2,3,4-tetrahydro-2,3-dioxo-5-quinoxalinyl)methyl]-phosphonic acid]; and 1-(phenanthrene-2-carbonyl)piperazine-2,3-dicarboxylic acid and the like.

Examples of miscellaneous drugs within the scope of the present disclosure that can be complexed with rosette nanotubes include nortriptyline, amytriptyline, fluoxetine (PROZAC®), paroxetine HCl (PAXIL®), trimipramine, oxcarbazepine (TRILEPTAL®), eperisone, misoprostol (a prostaglandin E₁ analog), latanoprost (a prostaglandin F₂ [symbol] analog) melatonin, and steroids (e.g., pregnenolone, triamcinolone acetonide, methylprednisolone, and other anti-inflammatory steroids) and the like.

Examples of antiviral drugs within the scope of the present disclosure that can be complexed with rosette nanotubes include Abacavir, Aciclovir, Acyclovir, Adefovir, Amantadine, Amprenavir, Ampligen, Arbidol, Atazanavir, Atripla (fixed dose drug), Boceprevir, Cidofovir, Combivir (fixed dose drug), Darunavir, Delavirdine, Didanosine, Docosanol, Edoxudine, Efavirenz, Emtricitabine, Enfuvirtide, Entecavir, Entry inhibitors, Famciclovir, Fixed dose combination (antiretroviral), Fomivirsen, Fosamprenavir, Foscarnet, Fosfonet, Fusion inhibitor, Ganciclovir, Ibacitabine, Immunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Stavudine, Synergistic enhancer (antiretroviral), Tea tree oil, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine and the like.

Examples of psychiatric drugs within the scope of the present disclosure that can be complexed with rosette nanotubes include Abilify, Adapin, Adartrel, Adderall, Alepam, Alertec, Aloperidin, Alplax, Alprax, Alprazolam, Alviz, Alzolam, Amantadine, Ambien, Amisulpride, Amitriptyline, Amoxapine, Amfebutamone, Anafranil, Anatensol, Ansial, Ansiced, Antabus, Antabuse, Antideprin, Anxiron, Apo-Alpraz, Apo-Primidone, Apo-Sertral, Aponal, Apozepam, Aripiprazole, Aropax, Artane, Asendin, Asendis, Asentra, Ativan, Atomoxetine, Aurorix, Aventyl, Axoren, Beneficat, Benperidol, Bimaran, Bioperidolo, Biston, Brotopon, Bespar, Bupropion, Buspar, Buspimen, Buspinol, Buspirone, Buspisal, Cabaser, Cabergoline, Calepsin, Calcium carbonate, Calcium carbimide, Calmax, Carbamazepine, Carbatrol, Carbolith, Celexa, Chloraldurat, Chloralhydrat, Chlordiazepoxide, Chlorpromazine, Cibalith-S, Cipralex, Citalopram, Clomipramine, Clonazepam, Clozapine, Clozaril, Concerta, Constan, Convulex, Cylert, Dapotum, Daquiran, Daytrana, Defanyl, Dalmane, Damixane, Demolox, Depad, Depakene, Depakote, Depixol, Desyrel, Dostinex, dextroamphetamine, Dexedrine, Diazepam, Didrex, Divalproex, Dogmatyl, Dolophine, properidol, Edronax, Efectin, Effexor (Efexor), Eglonyl, Einalon S, Elavil, Elontril, Endep, Epanutin, Epitol, Equetro, Escitalopram, Eskalith, Eskazinyl, Eskazine, Etrafon, Eukystol, Eunerpan, Faverin, Fazaclo, Fevarin, Finlepsin, Fludecate, Flunanthate, Fluoxetine, Fluphenazine, Flurazepam, Fluspi, Fluspirilen, Fluvoxamine, Focalin, Gabapentin, Geodon, Gladem, Glianimon, Halcion, Halomonth, Haldol, Haloperidol, Halosten, Imap, Imipramine, lmovane, JJanimine, Jatroneural, Kalma, Keselan, Klonopin, Lamotrigine, Largactil, Lecital, Levomepromazine, Levoprome, Leponex, Lexapro, Libritabs, Librium, Linton, Liskantin, Lithane, Lithium, Lithizine, Lithobid, Lithonate, Lithotabs, Lorazepam, Loxapac, Loxapine, Loxitane, Ludiomil, Lunesta, Lustral, Luvox, Lyrica, Lyogen, Manegan, Manerix, Maprotiline, Mellaril, Melleretten, Melleril, Melneurin, Melperone, Meresa, Mesoridazine, Metadate, Methamphetamine, Methotrimeprazine, Methylin, Methylphenidate, Minitran, Mirapex, Mirapexine, Moclobemide, Modafinil, Modalina, Modecate, Moditen, Molipaxin, Moxadil, Murelax, Myidone, Mylepsinum, Mysoline, Nardil, Narol, Navane, Nefazodone, Neoperidol, Neurontin, Nipolept, Norebox, Normison, Norpramine, Nortriptyline, Novodorm, Olanzapine, Omca, Oprymea, Orap, Oxazepam, Pamelor, Parnate, Paroxetine, Paxil, Peluces, Pemoline, Pergolide, Permax, Permitil, Perphenazine, Pertofrane, Phenelzine, Phenyloin, Pimozide, Piportil, Pipotiazine, Pragmarel, Pramipexole, Pregabalin, Primidone, Prolift, Prolixin, Promethazine, Prothipendyl, Protriptyline, Provigil, Prozac, Prysoline, Psymion, Quetiapine, Ralozam, Reboxetine, Resimatil, Restoril, Restyl, Requip, Rhotrimine, Risperdal, Risperidone, Rispolept, Ritalin, Rivotril, Ropark, Ropinerole, Rubifen, Rozerem, Sediten, Seduxen, Selecten, Serax, Serenace, Serepax, Serenase, Serentil, Seresta, Serlain, Serlift, Seroquel, Seroxat, Sertan, Sertraline, Serzone, Sevinol, Sideril, Sifrol, Sigaperidol, Sinequan, Sinqualone, Sinquan, Sirtal, Solanax, Solian, Solvex, Songar, Stazepin, Stelazine, Stilnox, Stimuloton, Strattera, Sulpiride, Sulpiride Ratiopharm, Sulpiride Neurazpharm, Surmontil, Symbyax, Symmetrel, Tafil, Tavor, Taxagon, Tegretol, Telesmin, Temazepam, Temesta, Temposil, Terfluzine, Thioridazine, Thiothixene, Thombran, Thorazine, Timonil, Tofranil, Tradon, Tramadol, Tramal, Trancin, Tranax, Trankimazin, Tranquinal, Tranylcypromine, Trazalon, Trazodone, Trazonil, Trialodine, Trevilor, Triazolam, Trifluoperazine, Trihexane, Trihexyphenidyl, Trilafon, Trimipramine, Triptil, Trittico, Troxal, Tryptanol, Ultram, Valium, Valproate, Valproic acid, Valrelease, Vasiprax, Venlafaxine, Vestra, Vigicer, Vivactil, Wellbutrin, Xanax, Xanor, Xydep, Zamhexyl, Zeldox, Zimovane, Zispin, Ziprasidone, Zolarem, Zoldac, Zoloft, Zolpidem, Zonalon, Zopiclone, Zotepine, Zydis, Zyprexa and the like.

Accordingly, the rosette nanotubes of the present disclosure have hollow channels that can be used for drug encapsulation. Rosette nanotubes are able to incorporate water-insoluble drugs into their tubular structures by hydrophobic interactions with the core whereas their hydrophilic outer surface can shield such hydrophobic drugs in a physiological environment for subsequent prolonged release (even into the cell). Rosette nanotubes can also be chemically functionalized with peptides such as Arg-Gly-Asp-Ser-Lys to deliver growth factors for healthy tissue regeneration, such as healthy bone in osteosarcoma patients, after the delivery of drugs to kill cancer cells.

A biologically acceptable medium includes, but is not limited to, any and all solvents, dispersion media, and the like which may be appropriate for the desired route of administration of the complexes of the present disclosure. The use of such media for pharmaceutically active substances is known in the art. Except insofar as any conventional media or agent is incompatible with the activity of the small molecule, protein, polypeptide and/or peptide, its use in the pharmaceutical preparation of the invention is contemplated. Suitable vehicles and formulations are described, for example, in the book Remington's Pharmaceutical Sciences (Remington's Pharmaceutical Sciences. Mack Publishing Company, Easton, Pa., USA 1985). These vehicles include injectable formulations.

The complexes of the present invention may be administered by any suitable route. For example, a pharmaceutical preparation may be administered in tablets or capsules, by injection, by infusion, by inhalation, topically (e.g., by lotion or ointment), by suppository, by controlled release patch, or the like.

As used herein, the terms “parenteral administration” and “administered parenterally” are intended to include, but are not limited to, modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal injection, intrasternal injection, infusion and the like.

As used herein, the terms “systemic administration,” “administered systemically,” “peripheral administration” and “administered peripherally” are intended to include, but are not limited to, the administration of a compound, drug or other material other than directly into the central nervous system, such that it enters an individual's system and, thus, is subject to metabolism and other like processes, such as, for example, subcutaneous administration.

The complexes described herein may be administered to an individual (e.g., a human or animal such as a non-human primate) for therapy by any suitable route of administration, including orally, nasally, rectally, intravaginally, parenterally, intracisternally, topically, buccally, sublingually and the like.

Regardless of the route of administration selected, the pharmaceutical compositions of the present invention are formulated into pharmaceutically acceptable dosage forms such as described below or by other conventional methods known to those of skill in the art. Actual dosage levels of the pharmaceutical compositions described herein may be varied so as to obtain an amount of the compound which is effective to achieve the desired therapeutic response for a particular patient, composition, and mode of administration, without being toxic to the patient.

The selected dosage level will depend upon a variety of factors including the activity of a particular compound or ester, salt or amide thereof, the route of administration, the time of administration, the rate of excretion of the particular compound being employed, the duration of the treatment, other drugs, compounds and/or materials used in combination with the particular complex employed, the age, sex, weight, condition, general health and prior medical history of the patient being treated, and like factors well known in the medical arts. A physician, veterinarian or research scientist having ordinary skill in the art can readily determine and prescribe the effective amount of the pharmaceutical composition required. For example, the physician, veterinarian or research scientist could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, doses of the compounds of this invention for a patient will range from about 0.0001 to about 100 mg per kilogram of body weight per day, or from about 0.001 to 30 mg/kg body weight, from about 0.01 to 25 mg/kg body weight, from about 0.1 to 20 mg/kg body weight, from about 1 to 10 mg/kg, from about 2 to 9 mg/kg, from about 3 to 8 mg/kg, from about 4 to 7 mg/kg, or from about 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of biologically active agent can include a single treatment or, preferably, can include a series of treatments. It will also be appreciated that the effective dosage for treatment may increase or decrease over the course of a particular treatment. Changes in dosage may result from the results of diagnostic assays.

If desired, the effective daily dose of the active compound may be administered as one, two, three, four, five, six or more sub-doses administered separately at appropriate intervals throughout the day, optionally, in unit dosage forms. In certain embodiments, an effective dose is given every other day, twice a week, once a week or once a month.

The term “treatment,” as used herein, is intended to include, but is not limited to, prophylaxis, therapy and cure. A patient or individual receiving treatment is any animal in need, such as humans, non-human primates, and other mammals such as horses, camels, cattle, swine, sheep, poultry, goats, rabbits, mice, guinea pigs, dogs, cats and the like.

A complex of the invention can be administered as such or in admixtures with pharmaceutically acceptable carriers and can also be administered in conjunction with other antimicrobial agents such as penicillins, cephalosporins, aminoglycosides, glycopeptides and the like. Conjunctive therapy includes sequential, simultaneous and separate administration of an active compound in such a way that the therapeutic effects of the first administered compound are still present when a subsequent administration is performed.

Another aspect of the present disclosure provides pharmaceutically acceptable compositions comprising a therapeutically effective amount of one or more of the complexes described herein, formulated together with one or more pharmaceutically acceptable carriers (additives) and/or diluents. As described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject complexes may be simply dissolved or suspended in sterile water.

As used herein, the term “therapeutically effective amount” is intended to include, but is not limited to, an amount of a compound, material, or composition comprising a complex of the present invention which is effective for producing a desired therapeutic effect in at least a subpopulation of cells in an animal and thereby altering (e.g., reducing or increasing) the biological consequences of one or more pathways in the treated cells, at a reasonable benefit/risk ratio.

As used herein, the term “pharmaceutically acceptable” is intended to include, but is not limited to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” is intended to include, but is not limited to, a pharmaceutically acceptable material, composition or vehicle, such as a liquid or solid filler, diluent, excipient, solvent or encapsulating material, involved in carrying or transporting the complexes of the present disclosure from one organ, or portion of the body, to another organ, or portion of the body. Each carrier must be “acceptable” in the sense of being compatible with the other ingredients of the formulation and not unduly dangerous to the patient. Examples of materials which can serve as pharmaceutically acceptable carriers include but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations, which could easily be determined by one of skill in the art.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in compositions of the present invention.

Examples of pharmaceutically acceptable antioxidants include but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the pharmaceutical art. The amount of active ingredient which can be combined with a carrier material to produce a single dosage form will vary depending upon the individual being treated and the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, from about 5 percent to about 70 percent, from about 10 percent to about 30 percent, from about 15 percent to about 25 percent, or from about 18 percent to about 22 percent. In an alternative embodiment, compounds of the present invention can be administered per se, i.e., in the absence of carrier material.

Methods of preparing the formulations or compositions of the present invention include the step of associating a complex described herein with a carrier and, optionally, one or more accessory ingredients. In general, the formulations are prepared by uniformly associating a complex of the present invention with liquid carriers, finely divided solid carriers, or both, and, optionally, shaping the product.

Formulations suitable for oral administration may be in the form of capsules, cachets, pills, tablets, lozenges (using a flavored basis, such as sucrose and acacia or tragacanth), powders, granules, as a solution or a suspension in an aqueous or non-aqueous liquid, as an oil-in-water or water-in-oil liquid emulsion, as an elixir or syrup, as pastilles (using an inert base, such as gelatin and glycerin, or sucrose and acacia) and/or as mouth washes and the like, each containing a predetermined amount of a complex of the present invention as an active ingredient. A complex of the present invention may also be administered as a bolus, electuary or paste.

Ointments, pastes, creams and gels may contain, in addition to a complex of the present disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a complex of the present disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a complex of the present invention to the body. Such dosage forms can be made by dissolving or dispersing the complex in the proper medium. Absorption enhancers can also be used to increase the flux of the complex across the skin. The rate of such flux can be controlled by either providing a rate controlling membrane or dispersing the complex in a polymer matrix or gel.

Pharmaceutical compositions of this invention suitable for parenteral administration comprise one or more complexes of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Injectable depot forms are made by forming microencapsule matrices of the complexes in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The present disclosure is directed to methods of forming a delivery complex, for example, by mixing one or more agents with fully formed rosette nanotubes or modules that self-assemble into rosette nanotubes, such as the compounds of formula I or formula II. According to one aspect, fully formed rosette nanotubes in the form of a powder is dissolved in water and heated to boiling. The solution is then cooled to room temperature. One or more agents is then added to the solution of nanotubes at a suitable temperature and for a suitable period of time until a complex of the nanotube and one or more agents forms. Suitable ratios of the nucleic acid to nanotube include about 0.01:1 (wt/wt) to about 1:0.1 (wt/wt).

The present invention also provides methods of treating diseases comprising using the complexes or compositions of the present invention. In particular, methods are provided for treating a patient having a disease, by administering to the patient a therapeutically effective amount of a complex or composition of the present invention. For in vivo therapies based on local injection (e.g., intratumoral, intramuscularly, into the peritoneal cavity, intracardiac, and aerosolized treatments) the RNT/small RNA complex is advantageously water soluble and so may be administered as an aqueous injection.

In accordance with certain examples, complexes of the present invention can be incorporated into pharmaceutical compositions suitable for administration. Such compositions typically comprise the complexes disclosed here and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In accordance with certain examples, a pharmaceutical composition of the invention is formulated to be compatible with its intended route of administration. Such pharmaceutical compositions may be administered by inhalation, transdermally, orally, rectally, transmucosally, intestinally, parenterally, intramuscularly, subcutaneously, intravenously or other suitable methods that will be readily selected by the person of ordinary skill in the art, given the benefit of this disclosure. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Examples of genetic and/or non-neoplastic diseases potentially treatable with the complex, compositions, and methods include, but are not limited to the following: adenosine deaminase deficiency; purine nucleoside phosphorylase deficiency; chronic granulomatous disease with defective p47phox; sickle cell with HbS, β-thalassemia; Faconi's anemia; familial hypercholesterolemia; phenylketonuria; ornithine transcarbamylase deficiency; apolipoprotein E deficiency; hemophilia A and B; muscular dystrophy; cystic fibrosis; Parkinsons, retinitis pigmentosa, lysosomal storage disease (e.g., mucopolysaccharide type 1, Hunter, Hurler and Gaucher), diabetic retinopathy, human immunodeficiency virus disease virus infection, acquired anemia, cardiac and peripheral vascular disease, osteoporosis and arthritis. In some of these examples of diseases, the therapeutic gene may encode a replacement enzyme or protein of the genetic or acquired disease, an antisense or ribozyme molecule, a decoy molecule, or a suicide gene product.

Cancers or neoplasms contemplated within the scope of the disclosure include, but are not limited to, carcinomas (i.e., malignant tumors derived from epithelial cells such as, for example, common forms of breast, prostate, lung and colon cancer), sarcomas (i.e., malignant tumors derived from connective tissue or mesenchymal cells), lymphomas (i.e., malignancies derived from hematopoietic cells), leukemias (i.e., malignancies derived from hematopoietic cells), germ cell tumors (i.e., tumors derived from totipotent cells. In adults most often found in the testicle or ovary; in fetuses, babies and young children, most often found on the body midline, particularly at the tip of the tailbone), blastic tumors (i.e., a typically malignant tumor which resembles an immature or embryonic tissue) and the like.

Examples of specific neoplasms intended to be encompassed by the present invention include, but are not limited to, acute lymphoblastic leukemia; myeloid leukemia, acute myeloid leukemia, childhood; adrenocortical carcinoma; AIDS-related cancers; AIDS-related lymphoma; anal cancer; appendix cancer; astrocytoma (e.g., cerebellar, cerebral); atypical teratoid/rhabdoid tumor; basal cell carcinoma; bile duct cancer, extrahepatic; bladder cancer; bone cancer, osteosarcoma and malignant fibrous histiocytoma; brain tumor (e.g., brain stem glioma, central nervous system atypical teratoid/rhabdoid tumors, central nervous system embryonal tumors, cerebellar astrocytoma, cerebral astrocytoma/malignant glioma, craniopharyngioma, ependymoblastoma, ependymoma, medulloblastoma, medulloepithelioma, pineal parenchymal tumors of intermediate differentiation, supratentorial primitive neuroectodermal tumors and/or pineoblastoma, visual pathway and/or hypothalamic glioma, brain and spinal cord tumors); breast cancer; bronchial tumors; Burkitt lymphoma; carcinoid tumor (e.g., gastrointestinal); carcinoma of unknown primary; central nervous system (e.g., atypical teratoid/rhabdoid tumor, embryonal tumors (e.g., lymphoma, primary); cerebellar astrocytoma; cerebral astrocytoma/malignant glioma; cervical cancer; chordoma; chronic lymphocytic leukemia; chronic myelogenous leukemia; chronic myeloproliferative disorders; colon cancer; colorectal cancer; craniopharyngioma; cutaneous T-cell lymphoma; embryonal tumors, central nervous system; endometrial cancer; ependymoblastoma; ependymoma; esophageal cancer; Ewing family of tumors; extracranial germ cell tumor; extragonadal germ cell tumor; extrahepatic bile duct cancer; eye cancer (e.g., intraocular melanoma, retinoblastoma); gallbladder cancer; gastric cancer; gastrointestinal tumor (e.g., carcinoid tumor, stromal tumor (gist), stromal cell tumor); germ cell tumor (e.g., extracranial, extragonadal, ovarian); gestational trophoblastic tumor; glioma (e.g., brain stem, cerebral astrocytoma); hairy cell leukemia; head and neck cancer; hepatocellular cancer; Hodgkin lymphoma; hypopharyngeal cancer; hypothalamic and visual pathway glioma; intraocular melanoma; islet cell tumors; Kaposi sarcoma; kidney cancer; large cell tumors; laryngeal cancer (e.g., acute lymphoblastic, acute myeloid); leukemia (e.g., acute myeloid, chronic lymphocytic, chronic myelogenous, hairy cell); lip and/or oral cavity cancer; liver cancer; lung cancer (e.g., non-small cell, small cell); lymphoma (e.g., AIDS-related, Burkitt, cutaneous Tcell, Hodgkin, non-Hodgkin, primary central nervous system); macroglobulinemia, Waldenstrom; malignant fibrous histiocytoma of bone and/or osteosarcoma; medulloblastoma; medulloepithelioma; melanoma; merkel cell carcinoma; mesothelioma; metastatic squamous neck cancer; mouth cancer; multiple endocrine neoplasia syndrome; multiple myeloma/plasma cell neoplasm; mycosis fungoides; myelodysplastic syndromes; myelodysplastic/myeloproliferative diseases; myelogenous leukemia (e.g., chronic, acute, multiple); myeloproliferative disorders, chronic; nasal cavity and/or paranasal sinus cancer; nasopharyngeal cancer; neuroblastoma; non-Hodgkin lymphoma; non-small cell lung cancer; oral cancer; oral cavity cancer, oropharyngeal cancer; osteosarcoma and/or malignant fibrous histiocytoma of bone; ovarian cancer (e.g., ovarian epithelial cancer, ovarian germ cell tumor, ovarian low malignant potential tumor); pancreatic cancer (e.g., islet cell tumors); papillomatosis; paranasal sinus and/or nasal cavity cancer; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma cell neoplasm/multiple myeloma; pleuropulmonary blastoma; primary central nervous system lymphoma; prostate cancer; rectal cancer; renal cell cancer; renal, pelvis and/or ureter, transitional cell cancer; respiratory tract carcinoma involving the nut gene on chromosome 15; retinoblastoma; rhabdomyosarcoma; salivary gland cancer; sarcoma (e.g., Ewing family of tumors, Kaposi, soft tissue, uterine); Sézary syndrome; skin cancer (e.g., non-melanoma, melanoma, merkel cell); small cell lung cancer; small intestine cancer; soft tissue sarcoma; squamous cell carcinoma; squamous neck cancer with occult primary, metastatic; stomach cancer; supratentorial primitive neuroectodermal tumors; T-cell lymphoma, cutaneous; testicular cancer; throat cancer; thymoma and/or thymic carcinoma; thyroid cancer; transitional cell cancer of the renal, pelvis and/or ureter; trophoblastic tumor; unknown primary site carcinoma; urethral cancer; uterine cancer, endometrial; uterine sarcoma; vaginal cancer; visual pathway and/or hypothalamic glioma; vulvar cancer; Waldenström macroglobulinemia; Wilms tumor and the like. For a review, see the National Cancer Institute's Worldwide Website (cancer.gov/cancertopics/alphalist). One of skill in the art will understand that this list is exemplary only and is not exhaustive, as one of skill in the art will readily be able to identify additional cancers and/or neoplasms based on the disclosure herein.

### EXAMPLES

The following examples are specific embodiments of the present invention but are not intended to limit it.

### Example 1

### Preparation of a Rosette Nanotubes

Rosette nanotubes formed from modules of formula I with lysine as a side chain (identified herein as K1) were prepared using the methods described in Fenniri, J. Am. Chem. Soc, 2001, 123, 3854-3855 incorporated by reference herein in its entirety. Rosette nanotubes formed from modules of formula II (identified herein as TBL) were prepared according to the methods described in Moralez, J. Am. Chem. Soc. 2005, 127, 8307-8309 hereby incorporated by reference in its entirety.

### Example 2

### Characterization of TAM-Loaded K1 and TBL

K1 building block (4.30 mg) was first dissolved in deuterated methanol (CD₃OD, 1 mL) and aged for 1 day. t-BuOH (3 μL) (Sigma, ≧99.5%, anhydrous) was added as an internal standard to quantify by ¹H-NMR the extent of TAM (tamoxifen, Sigma, T5648) association with the RNTs. TAM (5 mg) was then added, thus resulting in a solution with a 5:3 TAM:K1 molar ratio. The solution was aged for two days to allow for the drug-loading process, and the supernatant was isolated for subsequent ¹H-NMR studies. Other solutions with 5:1 and 5:5 TAM:K1 molar ratios were also prepared. TBL building block (3 mg) was dissolved in CD₃OD (1 mL) and the same procedure was followed to prepare mixtures with TAM (1.53 mg) resulting in a solution with a 5:5 molar ratio of TAM:TBL.

The supernatants were characterized by DOSY NMR and UV-Vis. ¹H-NMR was used to observe the amount of drug loaded into the RNTs at various time points. All the NMR experiments were conducted on a Varian Direct Drive 600 MHz spectrometer with a dual broadband probe. UV-vis spectra were recorded on an Agilent 8453. After this series of spectral analyses were completed, CD₃OD was evaporated using a stream of nitrogen and replaced with the same volume of deuterated water (D₂O). Any hydrophobic TAM that was not incorporated into the nanotubes precipitated in D₂O and was filtered out prior to NMR studies.

The NMR peaks corresponding to the drug were monitored and their integration was compared to the internal standard to determine the amount of encapsulated versus free drug in CD₃OD. Under the same experimental conditions, DOSY NMR was performed to detect subtle changes in the diffusion coefficients of TAM in the presence of K1 and TBL.

For UV-Vis experiments, the supernatants of the drug-loaded K1 and TBL were diluted with methanol to a final concentration of 25 μg/mL of TAM. In addition, three other solutions were prepared, ie, 5 μg/mL TAM, 25 μg/mL K1, and 25 μg/mL TBL. Each solution (3 mL) was placed in a 1 cm×1 cm cuvette for UV-Vis experiments. In another series of experiments the solutions were prepared at the same concentration in D₂O and analyzed by UV-Vis spectroscopy. Because TAM was insoluble in D₂O, no corresponding peak was recorded. Peaks from TAM and TAM-K1 were compared with those from TAM and TAM-TBL.

Because of their large molecular weight and long relaxation time, K1 and TBL were not detected by ¹H-NMR spectroscopy. Thus as the drug becomes encapsulated by the RNTs, its integration by ¹H-NMR decreases relative to the internal standard (t-BuOH). At the TAM: K1 molar ratio of 5:3 (Table 1 below), the incorporation of the drug reached an average maximum of 22% within one day (ca. 0.254 mg of TAM per 1 mg of K1).

TABLE 1
¹H-NMR spectroscopy of TAM-K1 mixture (5:3 molar ratio) at different
time intervals monitored at 7.25 ppm.

| Time (h) | | | | | | | |
|---|---|---|---|---|---|---|---|
| 0 | 1 | 2 | 3 | 4 | 5 | 24 | 48 |

| | | | | | | | | |
|---|---|---|---|---|---|---|---|---|
| ¹H integration | 2.20 | 2.18 | 2.09 | 2.07 | 2.02 | 2.0 | 1.69 | 1.68 |

Results also showed that more TAM was incorporated as additional K1 was added (Table 2 below).

TABLE 2
¹H-NMR spectroscopy of TAM-K1 and TAM-TBL at
different molar ratios monitored at 2.68-3 ppm.

| | TAM-K1 molar ratio | | | |
|---|---|---|---|---|
| | (5:1) | (5:3) | (5:5) | (5:5) |
| ¹H integration of TAM | 0.03 | 2.33 | 2.33 | 1 |
| before encapsulation | | | | |
| ¹H integration of TAM | 0.02 | 1.85 | 1.72 | 0.72 |
| after encapsulation | | | | |
| Average change | 2.13% | 22% | 26.5% | 30% |

Interestingly, the more stable and more hydrophobic TBL incorporated a slightly larger amount of TAM relative to K1 (11.3%±2.1% greater loading), as shown in Table 2. To provide further evidence for the incorporation of TAM into K1 and TBL, changes in the diffusion coefficient of TAM obtained from the DOSY NMR experiments (Tables 3 and 4 below) were measured and were found to vary from 7.27 to 5.37 for K1, and from 7.27 to 5.48 (for TBL). This reduction in diffusion coefficient is indicative of an interaction between the drug and the large RNT assemblies.

TABLE 3
DOSY NMR of TAM and of TAM-K1 mixture (5:5 molar ratio).

| | TAM peaks | | | | | | |
|---|---|---|---|---|---|---|---|
| | Peak 1 | Peak 2 | Peak 3 | Peak 4 | Peak 5 | Peak 6 | Average |
| Chemical shift (ppm) | 6.7 | 6.55 | 3.9 | 3.1 | 2.58 | 2.41 | — |
| Diffusion Coefficient | 7.2 | 7.4 | 7.2 | 7.4 | 7.4 | 7 | 7.27 |
| (m²/s) before | | | | | | | |
| Diffusion Coefficient | 5.4 | 5.4 | 5.6 | 5.6 | 5.2 | 5.2 | 5.37 |
| (m²/s) after | | | | | | | |

TABLE 4
DOSY NMR of TAM and of TAM-TBL mixture (5:5 molar ratio).

| | TAM peaks | | | | | | |
|---|---|---|---|---|---|---|---|
| | Peak 1 | Peak 2 | Peak 3 | Peak 4 | Peak 5 | Peak 6 | Average |
| Chemical shift (ppm) | 6.7 | 6.55 | 3.9 | 3.1 | 2.58 | 2.41 | — |
| Diffusion Coefficient | 7.2 | 7.4 | 7.2 | 7.4 | 7.4 | 7 | 7.27 |
| (m²/s) before | | | | | | | |
| Diffusion Coefficient | 5.3 | 5.4 | 5.6 | 5.6 | 5.6 | 5.4 | 5.48 |
| (m²/s) after | | | | | | | |

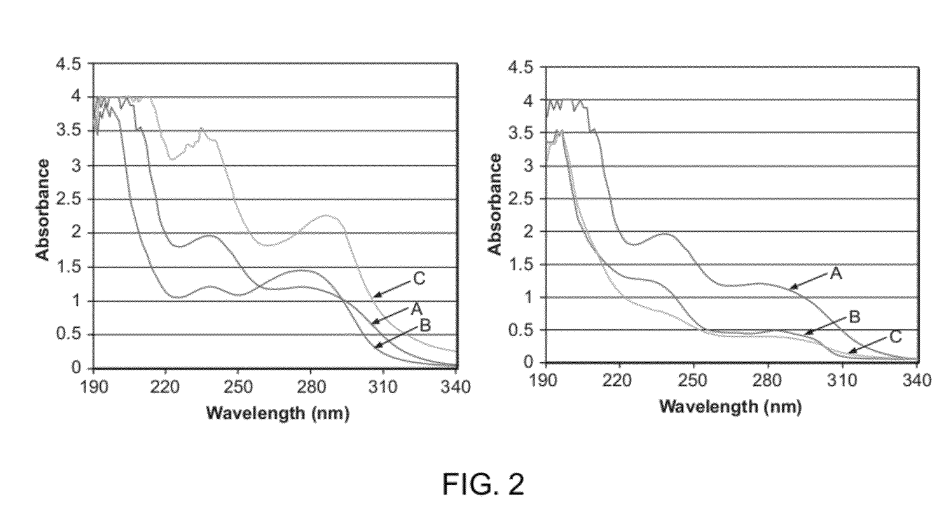

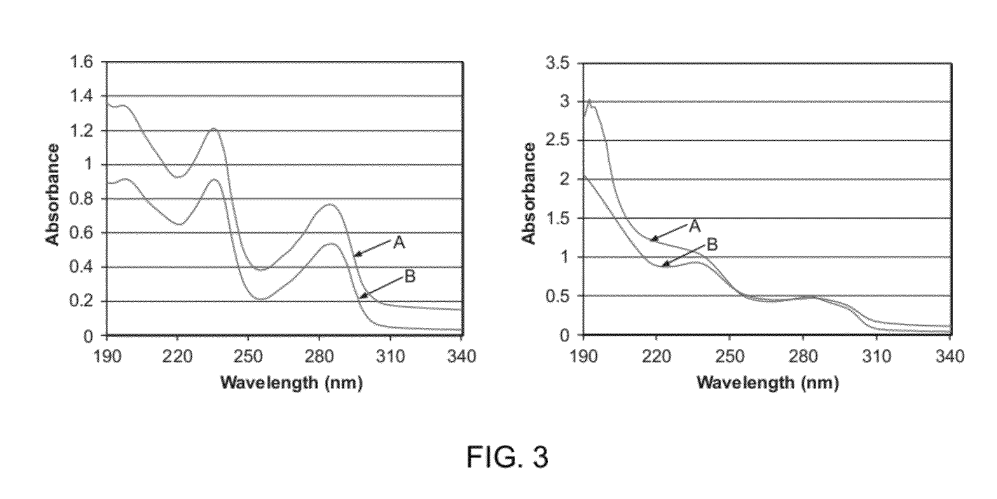

Incorporation of TAM in K1 and TBL was also investigated by UV-Vis experiments (FIG. 2 and FIG. 3), which showed significantly different profiles for TAM-K1 and TAM-TBL in methanol. When the UV-Vis spectra were recorded in water, which mimics the actual physiological environment in the body, the UV-Vis profiles of K1 and TBL were significantly different from those of TAM-K 1 and TAM-TBL, respectively. This result indicated once again that TAM do bind to the RNTs.

### Example 3

### Sample Preparation of TAM-Loaded K1 and TBLs for AFM

For atomic force microscopy (AFM) experiments, samples prepared in D₂O were diluted to 25 μg/mL and imaged using a Digital Instruments/Veeco Instruments MultiMode Nanoscope IV AFM equipped with an E scanner. Height profiles were measured using silicon cantilevers (MikroMasch USA, Inc.) with low spring constants of 4.5 N/m in tapping mode. To obtain a clear image of the surface, a low scan rate (0.5-1 Hz) and amplitude setpoints (1 V) were chosen during measurements.19 Clean mica substrates (Mica-Grade V-4 SPI, Catalog number 01918-CF and Lot number 1100315) were prepared and the samples were deposited by spin-coating 20 μL of each solution at 2000 rpm for 20 s.

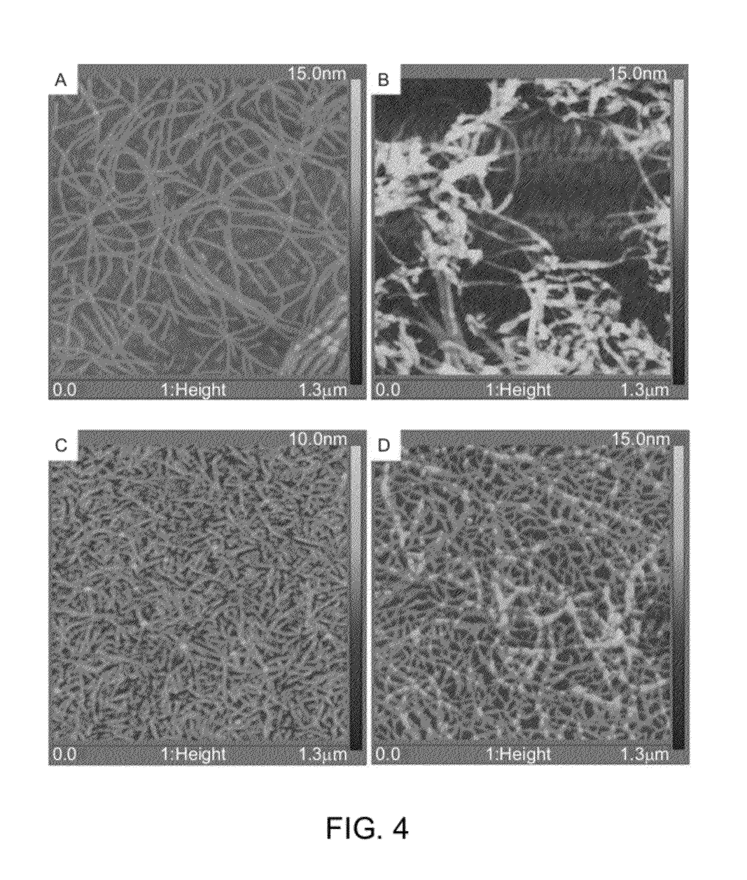

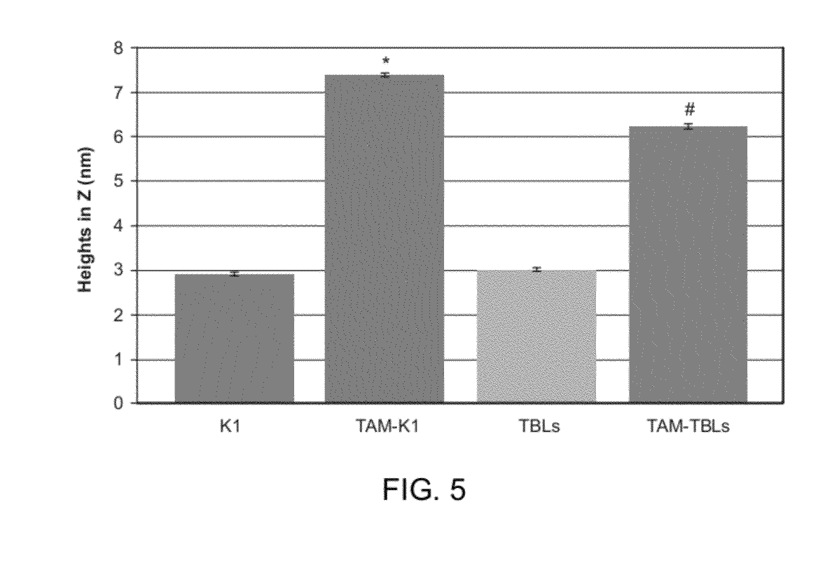

Tapping mode AFM (FIG. 4) showed different heights for the aqueous solutions of RNTs in the presence and in the absence of TAM. Height measurements showed a significantly greater value for RNT-encapsulated TAM (FIG. 5). The average heights were 2.91 nm, 7.40 nm, 3.02 nm, and 6.22 nm for K1, TAM-K1, TBL, and TAM-TBL, respectively. While the values for the complexes are dramatically high relative to the uncomplexed RNTs, they do support the existence of an interaction between drug and RNTs (whose exact nature is still unknown at this stage).

### Example 4

### Discussion

The drug loading capacity of K1 and TBL was investigated under various conditions using ¹H-NMR. For equivalent concentrations of TAM and RNTs, ca. 30% of the hydrophobic anticancer drug was incorporated. The amount of RNTs present affected how much TAM could be loaded. Successful TAM loading was further confirmed by DOSY NMR experiments where diffusion coefficients of TAM diminished significantly after interacting with the RNTs. UV-Vis experiments supported the presence of an interaction between TAM and RNTs. Without wishing to be bound by theory, the TAM molecules may be incorporated into the RNTs by intercalating between the ĜC bases given the hypochromic effect observed in the UV-Vis spectra of TAM-TBL. Furthermore, AFM height profiles showed a dramatic increase in RNTs cross-section as a result of interaction with TAM. It should also be noted the RNTs maintained their structural integrity after TAM loading.

Therefore, compared with conventional drug delivery systems, RNTs and TBLs as described here can self-assemble in situ and are water-soluble, biocompatible nanomaterials, suitable for hydrophobic drug incorporation. Although the p[resent disclosure describes the ability of RNTs to load TAM in water, one of skill in the art will readily recognize that other drugs and hydrophobic drugs in particular can be bound to or encapsulated by RNTs for use as delivery agents.

### Example 5

### Characterization of DEX-Loaded K1 and TBL

¹H and DOSY NMR studies. To test the ability of K1 to deliver hydrophobic drugs, 4.07 mg of K-ĜC were dissolved in deuterated methanol (CD₃OD, 1 mL) and aged for 1 day at room temperature. Tert-butanol (t-BuOH, 1.3 μL, Sigma, ≧99.5% anhydrous) was added as a ¹H NMR internal standard to quantify the extent of DEX interaction with K1. DEX (dexamethasone, 5.11 mg, Sigma, D4902) was then added resulting in a solution composed of DEX:K1 with a molar ratio of 5:3. The solution was aged for 2 days prior to ¹H NM and DOSY NMR investigations. Proton NMR spectra and DOSY were recorded on a Varian Direct Drive VNMRS 600 spectrometer operating at a magnetic field strength of 14.1 T (600 MHz frequency). A dual broadband probe was used. ¹H NMR spectra were acquired at room temperature using a single pulse excitation with a 45° flip angle of 3.6 μs and an acquisition time of 1.7 s. The repetition time was 1.0 s. It should be noted that as DEX interacts with the RNTs, the corresponding proton integration and diffusion coefficient decrease proportionally as a result of the large relaxation time of the self-assembled RNTs.

Atomic force microscopy (AFM). AFM experiments were carried out on a Digital InstrumentsNeeco Instruments MultiMode Nanoscope IV AFM equipped with an E scanner. Silicon cantilevers (MikroMasch USA, Inc.) with low spring constants of 4.5 N/m were used in tapping mode with a scan rate of 0.5-1 Hz and amplitude setpoint of 1 V. K1 (20 μL of a 50-250 μg/mL solution) with/without DEX were spin-coated on freshly cleaved mica at 2,000 rpm for 20 s and dried in air prior to imaging.

UV-Vis spectroscopy. For UV-Vis experiments (Agilent 8453), the DEX:K1 (5:3 molar ratio) stock solution containing 4.07 mg of the drug-loaded RNTs in methanol (see above) was diluted to 25 μg/mL with methanol. Solutions of DEX (5 μg/mL) and K1 (25 μg/mL) in methanol were prepared, as well as the same three solutions in dH₂O.

DEX loading and release studies. Glass coverslips (Fisher Scientific, circular; diameter, 18 mm; thickness, 1 mm) were cleaned with methanol, acetone and water in a sonicator. Three groups were prepared in dH₂O: (a) glass coverslips were dipped in a water-soluble DEX solution (1 mg/mL, Sigma, D2915); (b) glass coverslips were first soaked in K1 solution (100 μg/mL) and after air-drying, they were then immersed into the water-soluble DEX solution (1 mg/mL); and (c) glass slides were dipped in the K1 solution (100 μg/mL) with 1 mg/mL water-soluble DEX. Then, all three groups were air-dried and incubated at 37° C. in PBS buffer (3 mL). Aliquots were then taken from the supernatant on a daily basis over a period of 9 days. The DEX solution was mixed with cupric ion in a pH 11 buffer. Because of the reduction of cupric ion, cuprous cation was produced and spectrophotometrically detected by incorporating with 2-(4-carboxyquinolin-2-yl) quinoline-4-carboxylic acid (Pierce Biotechnology). DEX concentrations were quantified by comparing with the standard curve.

Contact angle measurements. To confirm the adsorption of K1 on the glass slides, the contact angles of water droplets were measured before and after coating with the K1 solution (100 μg/mL) using a static contact angle meter (KRUSS, FM40, Germany). An accurate auto pipette was used to ensure that the volume of the sample (20 μL) remained constant across glass slides. The contact angles were measured ca. 30 s after the droplets were placed on the surfaces.

Bioactivity. Osteoblasts (bone-forming cells, ATCC, CRL-11372 population numbers <9) were seeded at 1,000 cells/cm² in well plates and were cultured in DMEM medium (Gibco/BRL, Grand Island, N.Y.) supplemented with 10% fetal bovine serum (FBS; HyClone, Logan, Utah) and 1% penicillin/streptomycin (P/S; Hyclone) under standard cell culture conditions (37° C., humidified, 5% CO₂/95% air environment). DEX-containing samples (10 μL) (see controlled release experiments) and freshly prepared and sterilized DEX (10⁻⁸ M) were added to the wells along with the cell culture medium. After the prescribed time period, non-adherent cells were removed by washing twice with phosphate buffered saline (PBS). At the end of 1, 3 and 5 days, osteoblasts were fixed with 10% normal buffered formalin (NBF; Fisher Scientific), stained with 4′,6-diamidino-2-phenylindole, dihydrochloride (DAPI; Invitrogen, Carlsbad, Calif.), and counted at five random fields of view for each well under a fluorescence microscope (Zeiss Axiovert 200M, Peabody).

Statistical analysis. Data were expressed as the standard error of the mean (SEM). Statistics were performed using a student's one-tailed t test, with p<0.05 being considered statistically significant.

### Example 6

### Discussion

DEX was chosen as a model drug because of its wide use in orthopedic applications since it is an anti-inflammatory agent and can control stem cell differentiation. Due to their large molecular weight and long relaxation time, K1 and K1-encapsulated DEX were not detectable by ¹H NMR spectroscopy. Therefore, the percentage of drug loading were inferred from differences in peak heights of the drug relative to an internal standard (1-BuOH). As shown in Table 1 (below), at the DEX:K-ĜC molar ratio of 5:3, ca. 32% of DEX was captured by K1.

TABLE 1
¹H NMR spectroscopy of DEX and RNT-encapsulated DEX.^{a}

| | DEX peaks | | | | | |
|---|---|---|---|---|---|---|
| | 1 | 2 | 3 | 4 | 5 | 6 |
| Chemical shift (ppm) | 7.39 | 6.28 | 6.07 | 4.59 | 4.24 | 3.08 |
| ¹H integration of DEX | 1.43 | 1.29 | 1.30 | 1.45 | 2.89 | 1.47 |
| before encapsulation | | | | | | |
| ¹H integration of DEX | 0.98 | 0.92 | 0.95 | 0.97 | 1.95 | 0.96 |
| after encapsulation | | | | | | |
| % Change | 32 | 29 | 27 | 33 | 33 | 35 |

^{a}The internal standard (t-BuOH) has a chemical shift of 1.22 ppm and an integration corresponding to 9 protons both before and after encapsulation of DEX. See supporting information section for NMR spectra.

In agreement with this result, a significant change in the diffusion coefficient of DEX was observed in the DOSY spectra (Table 2 below).

TABLE 2
DOSY NMR of DEX only and RNT-encapsulated DEX.

| | DEX peaks | | | | | | |
|---|---|---|---|---|---|---|---|
| | | | | | | | Aver- |
| | 1 | 2 | 3 | 4 | 5 | 6 | age |
| Chemical shift (ppm) | 7.39 | 6.28 | 6.07 | 4.59 | 4.24 | 3.08 | — |
| Diffusion Coefficient | 5.6 | 5.8 | 5.8 | 5.8 | 6 | 5.8 | 5.8 |
| (m²/s) of DEX before | | | | | | | |
| encapsulation | | | | | | | |
| Diffusion Coefficient | 5 | 5 | 5 | 5.2 | 5.4 | 5 | 5.1 |
| (m²/s) of DEX before | | | | | | | |
| encapsulation | | | | | | | |

^{a}The internal standard (t-BuOH) has a chemical shift of 1.22 ppm and an integration of corresponding to 9 protons both before and after encapsulation of DEX. See supporting information section for NMR spectra.

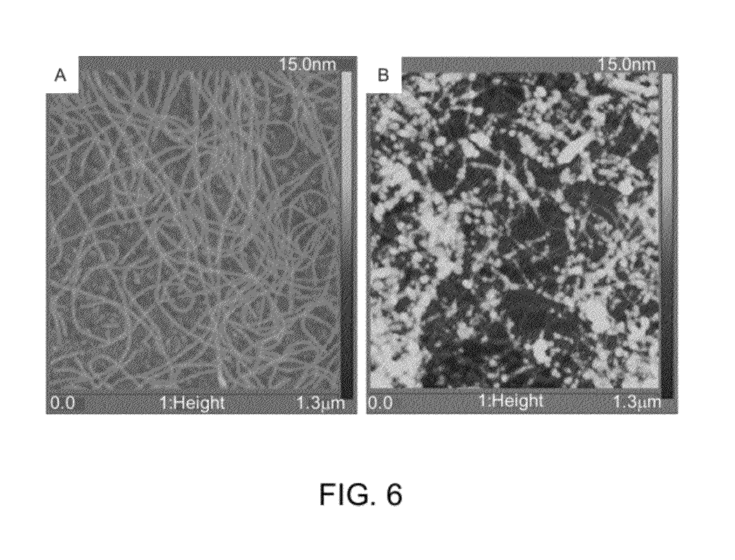

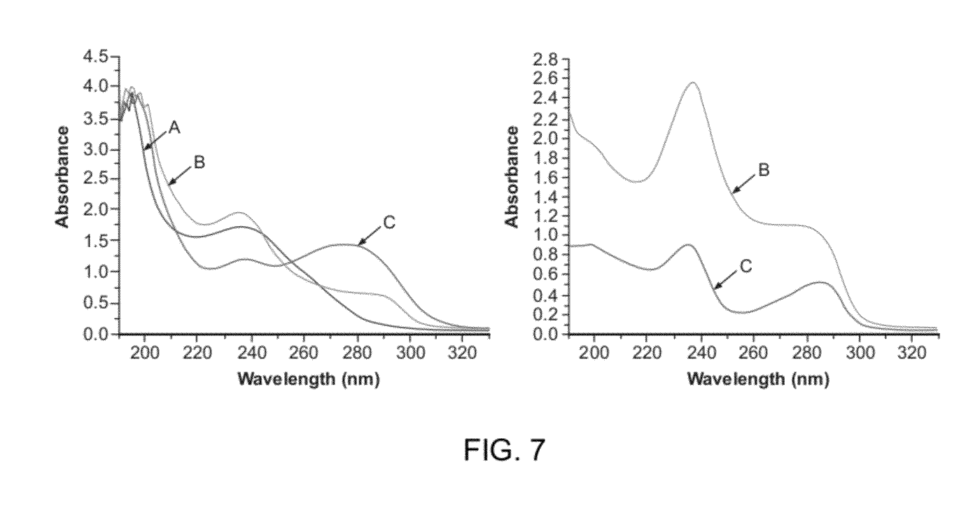

Tapping mode atomic force microscopy (AFM) showed significantly different height profiles for K1 before and after incorporation of DEX (FIG. 6). The UV-Vis spectra recorded in methanol showed a significant hypochromic effect at 280 nm and a hyperchromic effect around 240 nm. In water, on the other hand, a dramatic hyperchromic effect was observed around 280 and 240 nm suggesting that DEX significantly alters the stacking interaction in K1 (FIG. 7). It should be noted that DEX is insoluble in water and that the only way that K1's UV-Vis spectrum may be altered is via the formation of a stable DEX-K1 complex possibly via intercalation/stacking interactions.

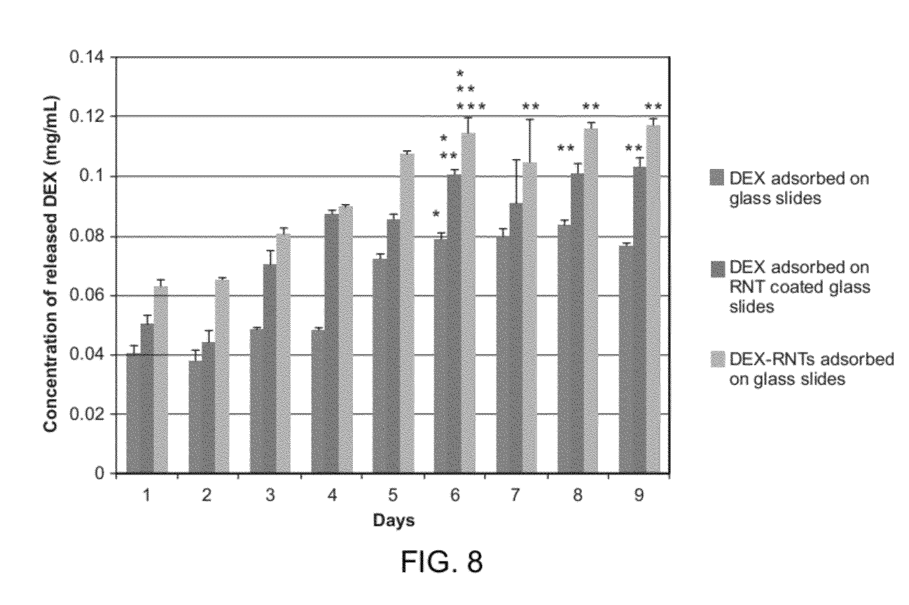

To understand how DEX is incorporated into K1 and to determine the DEX loading and release behavior, the DEX-K1 complex was prepared using two different methods. The first approach consisted of physically adsorbing K1 onto a glass slide and then dipping the slide in a water-soluble DEX solution. The second approach consisted of assembling K1 in the water-soluble DEX solution and then adsorbing the complex on a glass slide. To confirm that K1 adhered to the glass slide surfaces, contact angle measurements were carried out. A notable decrease in contact angles (from 54.1±2.1 to 43.9±3.5) for the RNT-coated slides was measured confirming that the hydrophilicity of the glass slide surface increased upon RNT adhesion. K1 prepared using the first approach retained ca. 24.8% more DEX than the control sample (uncoated glass slide dipped in the water-soluble DEX solution) and released DEX up to 6 days. Using the second approach, K1 incorporated ca. 42% more drugs than the control sample (FIG. 8).

These results along with the UV-Vis, AFM, and NMR data suggest that DEX was incorporated into K1 in the process of self-assembly by hydrophobic and base stacking interactions. Thus, for orthopedic applications, RNTs not only enhance osteoblast adhesion and subsequent functions (such as calcium deposition) but can also incorporate drugs (such as DEX) and release them over an extended period of time.

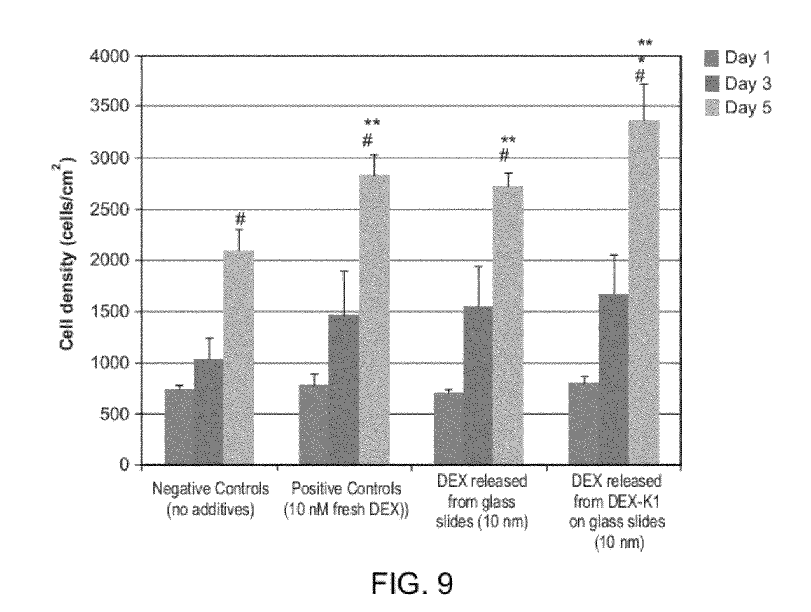

Equally important is the biological activity of the released DEX. Since DEX has been reported to increase fully differentiated cell density, osteoblast cell cultures with no additives (negative controls), commercially available water-soluble DEX (positive controls), released DEX from uncoated glass slides, and released DEX from the DEX-K1-coated glass slides were investigated. As shown in FIG. 9, osteoblast densities from the three groups with DEX were higher relative to the negative controls (without DEX). These results demonstrated that DEX released from DEX-K1-coated glass slides not only exerted the same biological effect as the drug released from DEX-coated glass slides but they also promoted higher osteoblast density.

Given the benefit of the above disclosure and description of exemplary embodiments, it will be apparent to those skilled in the art that numerous alternative and different embodiments are possible in keeping with the general principles of the invention disclosed here. Those skilled in this art will recognize that all such various modifications and alternative embodiments are within the true scope and spirit of the invention. While the invention has been illustrated and described in detail in the drawings and foregoing description, such illustration and description is to be considered as exemplary and not restrictive in character, it being understood that, only the preferred embodiments have been shown and described and that all changes and modifications that come within the spirit of the invention are desired to be protected. The appended claims are intended to cover all such modifications and alternative embodiments. It should be understood that the use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

What is claimed is:

1. A method of delivering a diagnostic or therapeutic agent to an individual comprising administering a complex of a rosette nanotube and one or more therapeutic or diagnostic agents.

2. The method of claim 1 wherein the nanotube is formed from the self-assembly of compounds having the formula 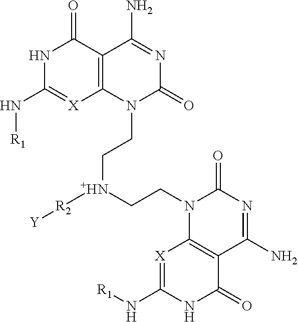
wherein X is CH or nitrogen; R₂ is hydrogen or a linker group; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such alkyl, straight or branched chain, saturated or unsaturated; and salts thereof.

3. The method of claim 3 wherein the nanotube is formed from the self-assembly of compounds having the formula 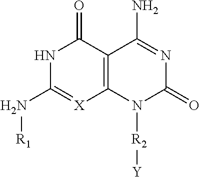
wherein X is CH or nitrogen; R₂ is hydrogen or a linker group; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such as alkyl, straight or branched chain, saturated or unsaturated; and salts thereof.

4. The method of claim 1 wherein the nanotube acts as a carrier for the therapeutic or diagnostic agent.

5. A method of treating an individual requiring treatment comprising administering a complex of a rosette nanotube and one or more therapeutic agents.

6. The method of claim 5 wherein the nanotube is formed from the self-assembly of compounds having the formula 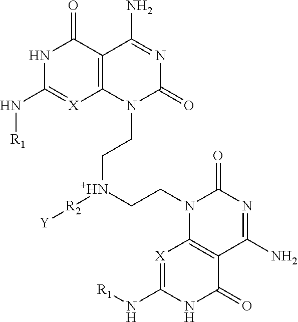
wherein X is CH or nitrogen; R₂ is hydrogen or a linker group; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such alkyl, straight or branched chain, saturated or unsaturated; and salts thereof.

7. The method of claim 5 wherein the nanotube is formed from the self-assembly of compounds having the formula 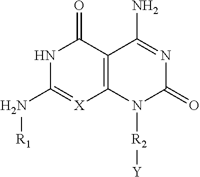
wherein X is CH or nitrogen; R₂ is hydrogen or a linker group; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such as alkyl, straight or branched chain, saturated or unsaturated; and salts thereof.

8. The method of claim 5 wherein the nanotube acts as a carrier for the therapeutic agent.

9. A method of making a delivery complex comprising
mixing together rosette nanotubes and one or more agents conditions which cause the rosette nanotubes to combine with the one or more agents.

10. A product made by the process comprising
mixing together rosette nanotubes and one or more agents in aqueous media under conditions which cause the rosette nanotubes to combine with the one or more agents.

11. A delivery complex comprising one or more agents and a rosette nanotube formed from the self assembly of compounds having the formula 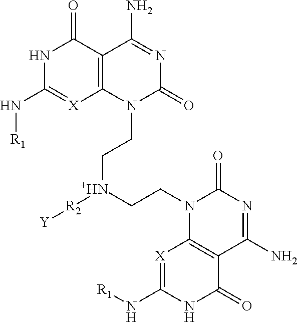
wherein X is CH or nitrogen; R₂ is hydrogen or a linker group; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such alkyl, straight or branched chain, saturated or unsaturated; and salts thereof.

12. A delivery complex comprising one or more agents and a rosette nanotube formed from the self assembly of compounds having the formula 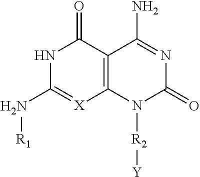
wherein X is CH or nitrogen; R₂ is hydrogen or a linker group; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such as alkyl, straight or branched chain, saturated or unsaturated; and salts thereof.

13. A delivery complex comprising one or more agents and a rosette nanotube formed from the self assembly in aqueous media of one or more compounds having the formula 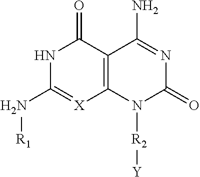
wherein X is CH or nitrogen; R₂ is hydrogen or a linker group; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such as alkyl, straight or branched chain, saturated or unsaturated; and salts thereof,
and one or more compounds having the formula 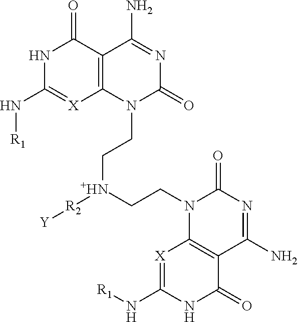
wherein X is CH or nitrogen; R₂ is hydrogen or a linker group; Y is absent when R₂ is hydrogen or is an amino acid or polypeptide having an amino group covalently bound to an α-carbon of the amino acid and the amino group is covalently bound to the linker group R₂; and R₁ is hydrogen or an aliphatic moiety, such alkyl, straight or branched chain, saturated or unsaturated; and salts thereof.
